(19) United States
(12) Patent Application Publication
(10) Pub. No.: US 2008/0234657 A1
(43) Pub. Date: Sep. 25, 2008

(54) METHODS FOR CONTRIBUTING TO CARDIOVASCULAR TREATMENTS

(75) Inventor: Julie Trudel, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/689,694

(22) Filed: Mar. 22, 2007

(51) Int. Cl. A61M 31/00

(52) U.S. Cl. 604/508

(57) ABSTRACT

Disclosed herein are methods that can contribute to cardiovascular treatments. The methods are designed to provide bioactive materials to the first proximal third of coronary arteries, the portion of these arteries most likely requiring treatment.

### FIELD OF THE INVENTION

The present invention relates to methods that can contribute to cardiovascular treatments. The methods are designed to provide bioactive materials to the first proximal third of coronary arteries, the portion of these arteries most likely requiring treatment.

### BACKGROUND OF THE INVENTION

For many years, it was believed that the main cause of heart attacks was the build-up of fatty plaque within coronary arteries. Sufficient plaque build-up narrows the affected artery to the point of drastically reducing or stopping blood flow, leading to a heart attack. While this scenario does occur, this type of blockage accounts only for about 30% of all heart attacks.

It is now believed that the majority of heart attacks are caused by the sudden rupture of “vulnerable plaque.” Vulnerable plaque does not build up within arteries to restrict blood flow but instead begins as fatty deposits within the inner lining of artery walls. Fatty deposits within artery walls can lead to local inflammation as immune cells fight the accumulation of these fatty deposits. The local inflammation takes the form of a growing atheromatous plaque within the vessel wall, generally separated from the bloodstream by a thin wall. When the inflammation of the atheromatous plaque reaches a critical point (sometimes reached due to additional inflammation caused by stress, hypertension, nicotine consumption, etc.), the vulnerable plaque can rupture, spilling its contents into the bloodstream. These vulnerable plaque contents are often too large to pass through affected vessels, obstructing blood flow to previously supplied tissues, and, in some instances, causing heart attacks. In addition, once vulnerable plaque ruptures, blood flow through the vessel may enter the inner tissue of the atheroma making the atheroma size suddenly increase, protruding into the lumen of the artery and narrowing or obstructing blood flow. Further, blood clotting on top of the site of the rupture can also become so large as to also block or obstruct the lumen of the artery, thereby stopping blood flow to the tissues the artery once supplied. Thus, the rupture of vulnerable plaque can lead to negative outcomes through several different mechanisms. Heart attacks caused by the rupture of vulnerable plaque present a difficult treatment problem because the presence of vulnerable plaque is not detectable through presently-used standard screening methods such as cardiac stress tests. Thus, methods that aid in the detection and/or treatment of vulnerable plaque are needed.

One specific opportunity for improvement in the treatment of heart attacks comes from recent clinical data demonstrating that acute coronary occlusions leading to heart attacks (including ST elevation myocardial infarctions (STEMIs)) are located in about the first third of coronary arteries. For instance, about 90% of left anterior descending artery (LAD) occlusions occur within about the first 40 mm of the LAD. Thus, treatments designed to specifically treat occlusions or vulnerable plaque in these areas could provide an important advance in the treatment or prevention of heart attacks. The present invention provides such treatment methods.

### SUMMARY OF THE INVENTION

Acute coronary occlusions leading to heart attacks are located in about the first third of coronary arteries. Treatments designed to deliver bioactive materials to these portions of the coronary arteries could provide an important advancement in the treatment and/or prevention of cardiovascular disease. Taking advantage of this opportunity, the present invention provides methods to treat vessel occlusions and/or vulnerable plaque in areas where they are most likely to occur. More specifically, the present invention provides methods to deliver bioactive materials to treat vessel occlusions and/or vulnerable plaque in the first third of coronary arteries.

Specifically, one embodiment according to the present invention includes a method of contributing to the treatment of cardiovascular disease comprising administering at least one bioactive material to a treatment site within about the first third of a coronary artery. In one embodiment, the coronary artery is the left anterior descending artery.

The administering can occur through, without limitation, implanting a form within about the first third of a coronary artery wherein the form releases bioactive materials and/or through the use of an injection catheter. When a form is implanted, the form can be one or more of, without limitation, a stent, a stent graft, a patch, a plurality of particles and a gel. Particularly useful bioactive materials to use in accordance with the present invention will include, without limitation, compounds selected from the group consisting of antianginal compounds, antiarrythmic compounds, antihypertensive compounds, antithrombotic compounds, blood-lipid lowering compounds, and combinations thereof. Bioactive materials used in accordance with the present invention can also, without limitation, inhibit P-selectin, prevent degranulation of mast cells, upregulate the insulin growth factor-1(IGF-1) receptor, upregulate IGF-1, downregulate death receptor 5 (DR5), lower blood lipids, stimulate the release of nitric oxide, and/or combinations thereof.

In a particular embodiment according to the present invention, the treatment site comprises vulnerable plaque.

In another embodiment, the form is comprised of a polymer having a characteristic selected from the group consisting of degradable, nondegradable, biodegradable, nonbiodegradable, bioerodible, nonbioerodable, bioadsorbable, nonbioadsorbable, controlled release, noncontrolled release, removable, nonremovable, and nonincompatible combinations thereof.

In yet another embodiment of the present invention, the form is comprised of particles and wherein the particles are coated with at least one antibody. In another embodiment, the at least one antibody is reactive with an antigen selected from the group consisting of αvβ3 integrin, P-selectin, ICAM-1, and fibrin and its related molecules such as, but not limited to, D-dimer.

### DEFINITION OF TERMS

Bioactive Material(s): As used herein “bioactive material” shall include any agent having a therapeutic effect in an animal. Exemplary, non limiting examples include a bioactive material can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials also can include drugs such as chemical or biological compounds that can have a therapeutic effect on a biological organism. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, anti-angiogenic agents, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers of the present invention.

Bioactive materials also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive materials within the intended meaning herein. Aspects of the present invention directed toward bioactive materials can include any or all of the foregoing examples.

Biodegradable: As used herein, “biodegradable” refers to a polymeric composition that is biocompatible and subject to being broken down in vivo through the action of normal biochemical pathways. From time to time bioresorbable and biodegradable may be used interchangeably, however they are not coextensive. Biodegradable polymers may or may not be reabsorbed into surrounding tissues, however all bioresorbable polymers are considered biodegradable. The biodegradable polymers of the present invention are capable of being cleaved into biocompatible byproducts through chemical- or enzyme-catalyzed hydrolysis.

Compatible: As used herein, “compatible” refers to a composition possessing the optimum, or near optimum combination of physical, chemical, biological and drug release kinetic properties suitable for a controlled-release coating made in accordance with the teachings of the present invention. Physical characteristics include durability and elasticity/ductility, chemical characteristics include solubility and/or miscibility and biological characteristics include biocompatibility. The drug release kinetic should be either near zero-order or a combination of first and zero-order kinetics.

Controlled release: As used herein, “controlled release” refers to the release of bioactive material from a medical device at a predetermined rate. Controlled release implies that the bioactive material does not come off the medical device surface sporadically or in an unpredictable fashion and additionally does not “burst” off of the device upon contact with a biological environment (also referred to herein as first order kinetics) unless specifically intended to do so. However, the term “controlled release” as used herein does not preclude a “burst phenomenon” associated with deployment. In some embodiments of the present invention an initial burst of bioactive material may be desirable followed by a more gradual release thereafter. The release rate may be steady state (commonly referred to as “timed release” or zero order kinetics), that is the bioactive material would be released in even amounts over a predetermined time (with or without an initial burst phase) or may be a gradient release. A gradient release implies that the concentration of bioactive material released from the medical device changes over time.

Copolymer: As used herein, a “copolymer” refers to a macromolecule produced by the simultaneous or step-wise polymerization of two or more dissimilar units such as monomers. Copolymer shall include bipolymers (two dissimilar units), terpolymers (three dissimilar units), etc.

Treatment: As used herein, the terms “treatment” or “contributing to the treatment of” include preventing, retarding the progression or growth of, shrinking, or eliminating a vessel occlusion or vulnerable plaque. As such, these terms include both medical therapeutic and/or prophylactic administration, as appropriate.

Treatment site: As used herein, the phrase “treatment site” includes any portion of a lumen that is intended to receive a beneficial or therapeutic effect of a bioactive material administered with the methods described herein. For example, the treatment site can be, without limitation, a site of an occlusion, a site comprising vulnerable plaque, a stenotic lesion in a blood vessel and/or a developing thrombus.

### DETAILED DESCRIPTION OF THE INVENTION

Acute coronary occlusions leading to heart attacks are often located in about the first third of coronary arteries. Treatments designed to deliver bioactive materials to these portions of the coronary arteries could provide an important advancement in the treatment and/or prevention of cardiovascular disease. The present invention includes methods designed to provide such treatments.

As will be understood by one of ordinary skill in the art, the methods according to the present invention can adopt a variety of forms to deliver bioactive materials to the first third of coronary arteries. For example, bioactive materials can be delivered with the use of bioactive material-eluting stents, stent grafts, patches, particles or gels. Bioactive materials can also be delivered with the use of appropriate delivery catheters. As such, methods according to the present invention can employ forms fabricated from synthetic and natural compositions of matter. Various embodiments can comprise one or more of carbon-based polymers, non carbon-based polymers, biodegradable polymers, metallic alloys, biodegradable metallic alloys and/or metallic alloys coated with various polymers. Further, and as will be understood by one of ordinary skill in the art, these materials can also comprise controlled release versions of the same materials.

Additionally, the polymer can have one or more characteristics including, but not limited to, degradable, nondegradable, biodegradable, nonbiodegradable, bioerodible, nonbioerodable, bioadsorbable, nonbioadsorbable, controlled release, noncontrolled release, removable, nonremovable, and nonincompatible combinations thereof.

The forms of the present invention are implanted within the proximal or first third of a coronary artery including, but not limited to, the right coronary artery (RCA), left anterior descending artery (LAD) and the left circumflex coronary artery (LCx). It will be understood that the term “proximal third” of the coronary artery will depend on the length of the artery in an individual patient. However, generally the proximal third of the coronary artery generally refers to about the first 50 mm of the RCA, about the first 40 mm of the LAD and about the first 25 mm of the LCx.

In one embodiment of the present invention, endoluminal bioactive material depots are fabricated to controllably release bioactive materials combating vulnerable plaque. In another embodiment, endoluminal paving compositions that controllably release bioactive materials combating vulnerable plaque are used. Endoluminal paving is a process whereby bioactive material eluting forms are percutaneously applied to blood vessels as endoluminal liners, resurfacing or ‘paving’, the underlying vascular wall. Depending on the type of form selected, endoluminal layers may function as, without limitation, wall supports, barriers, and/or therapeutic biomaterials or depots for local sustained bioactive material delivery.

In another embodiment of the present invention, the endoluminal bioactive material depots are particles. The drug-eluting particles can be administered to the patient systemically or locally such that the particles are targeted to the site of vulnerable plaque. In order to target the particles, the particles can be coated with antibodies reactive with the vulnerable plaque where they elute the bioactive material over time.

Antibodies suitable for use as targeting agents can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, including Fab, Fv and F(ab′)₂, Fab′, and the like. In addition, aggregates, polymers, and conjugates of immunoglobulins or their fragments can be used where appropriate so long as binding affinity for a particular antigen is maintained.

Antibodies can be associated with the particles by incorporating the antibodies into the polymer forming the particles, covalently binding the antibodies directly to the particle surface or indirectly associating the antibody with the particle, such as using a linker molecule covalently bound to the particle surface and the antibody attached to the linker.

Exemplary antibodies useful for directing particles to a site of vulnerable plaque include antibodies to the αvβ3 (alphavbeta3) integrin, fibrin and its related molecules such as, but not limited to, D-dimer, P-selectin and ICAM-1.

Bioactive materials that can be used in accordance with the present invention include, without limitation, angiotensin-converting enzyme (ACE) inhibitors, steroids, hormones, genetic material, antianginial drugs, anti-angiogenic agents, antiarrythmic agents, antihypertensive agents, antithrombotic drugs, blood-lipid lowering agents, drugs combating congestive heart failure, nitric oxide releasing compositions, and combinations thereof. Bioactive materials can also be used to, without limitation, decrease inflammation, inhibit P-selectin, prevent degranulation of mast cells, upregulate the insulin-like growth factor 1 (IGF-1) receptor and IGF-1, and/or down regulate death receptor 5 (DR5).

The bioactive material and the polymeric carrier may form a homogeneous matrix, or the bioactive material may be encapsulated in some way within the polymer. In their simplest form, the bioactive agent-eluting form consists of a dispersion of the bioactive material in a polymer matrix. The bioactive material is typically released as the polymeric matrix biodegrades in vivo into soluble products that can be excreted from the body. The bioactive material may be first encapsulated in a microsphere and then combined with the polymer in such a way that at least a portion of the microsphere structure is maintained. Alternatively, the bioactive material may be sufficiently immiscible in the polymer that it is dispersed as small droplets, rather than being dissolved, in the polymer. Either form is acceptable, but it is preferred that, regardless of the homogeneity of the composition, the release rate of the bioactive material in vivo remains controlled.

Non-limiting examples of polymers that may be especially useful in creating particular forms of the present invention can include poly-lactic acid (PLA); poly-glycolic acid (PGA), polycarbonates, polyurethanes, polycaprolactone and polyorthoester. Other polymers that may be used as appropriate include, without limitation, rapidly bioerodible polymers such as, without limitation, poly[lactide-co-glycolide], polyanhydrides, and polyorthoesters, whose carboxylic groups are exposed on the external surface as their smooth surface erodes. In addition, polymers containing labile bonds, such as, without limitation, polyanhydrides and polyesters can also be used. Representative natural polymers that can be used include, without limitation, proteins, such as zein, modified zein, casein, gelatin, gluten, serum albumin, or collagen, and polysaccharides, such as, without limitation, cellulose, dextrans, polyhyaluronic acid, polymers of acrylic and methacrylic esters and alginic acid. Representative synthetic polymers that can be used in accordance with the present invention include, without limitation, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Synthetically modified natural polymers that can be used in accordance with the present invention include, without limitation, alkyl celluloses, hydroxyalkyl celluloses, cellulose ethers, cellulose esters, and nitrocelluloses. Other polymers that can be used in accordance with the present invention include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof. These described polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques. Additionally biostable hydrogels, such as PEG-acrylate polymers are suitable for use in the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on the described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of contributing to the treatment of cardiovascular disease comprising administering at least one bioactive material to a treatment site within about the first third of a coronary artery.

2. A method according to claim 1 wherein said administering occurs by implanting a form within about the first third of said coronary artery wherein said form releases at least one bioactive material.

3. A method according to claim 2 wherein said form is selected from the group consisting of a stent, a stent graft, a patch, particles and a gel.

4. A method according to claim 1 wherein said administering occurs by delivering bioactive materials to said treatment site with the use of an injection catheter.

5. A method according to claim 1 wherein said at least one bioactive material comprises a compound selected from the group consisting of an antianginal compound, an antiarrythmic compound, an antihypertensive compound, an antithrombotic compound, a blood-lipid lowering compound, and combinations thereof.

6. A method according to claim 1 wherein said at least one bioactive material performs a function selected from the group consisting of decreasing inflammation, inhibiting P-selectin, preventing degranulation of mast cells, upregulating the insulin growth factor-1 (IGF-1) receptor, upregulating IGF-1, downregulating death receptor 5 (DR5), lowering blood lipids, stimulating the release of nitric oxide, and combinations thereof.

7. A method according to claim 1 wherein said treatment site comprises vulnerable plaque.

8. A method according to claim 2 wherein said form is comprised of a polymer having a characteristic selected from the group consisting of degradable, nondegradable, biodegradable, nonbiodegradable, bioerodible, nonbioerodable, bioadsorbable, nonbioadsorbable, controlled release, noncontrolled release, removable, nonremovable, and nonincompatible combinations thereof.

9. A method according to claim 1 wherein said coronary artery is the left anterior descending artery.

10. A method according to claim 3 wherein said particles are coated with at least one antibody.

11. A method according to claim 10 wherein said antibody is reactive with an antigen selected from the group consisting of αvβ3 integrin, P-selectin, ICAM-1, fibrin and D-dimer.
